(19) United States
(12) Patent Application Publication
(10) Pub. No.: US 2007/0276209 A1
(43) Pub. Date: Nov. 29, 2007

(54) Blood-Sugar Level Measuring Device

(76) Inventors: Fumiaki Emoto, Kanagawa (JP); Chiyohiro Hoshikawa, Ehime (JP); Tetsuo Hiraga, Tokushima (JP); Hiroshi Nakayama, Osaka (JP)

Correspondence Address: MCDERMOTT WILL & EMERY LLP, 600 13TH STREET, NW, WASHINGTON, DC, 20005-3096, US

(21) Appl. No.: 11/547,171

(22) PCT Filed: Apr. 22, 2005
(86) PCT No.: PCT/JP05/07728

§ 371 Date: Oct. 2, 2006

(30) Foreign Application Priority Data
Apr. 30, 2004 (JP) 2004-136510

(51) Int. Cl. A61B 5/05

(52) U.S. Cl. 600/319

(57) ABSTRACT

There is provided a mobile blood-sugar level monitoring device enabling a diabetic patient to accurately and easily perform management of blood-sugar level measurement timing, management of changes in blood-sugar level, and further prediction of a future blood-sugar level. The mobile blood-sugar level monitoring device having the function to measure the blood-sugar level and manage the obtained measurement data is provided with a data arithmetic-processing unit for arithmetically processing the measurement data by a predetermined method, displaying the result of the arithmetic process, and notifying measurement-timing for the blood-sugar level and a predicted blood-sugar level to the diabetic patient.

### TECHNICAL FIELD

The present invention relates to blood-sugar level monitoring devices, in which a blood-sugar level of a diabetic patient can be measured, and the measurement data of the blood-sugar level can be managed for an easy usage by diabetic patients and medical doctors.

### BACKGROUND ART

Recently, the number of diabetic patients is rapidly increasing. Diabetes is a metabolic disorder characterized by hyperglycemia caused by insulin secretory dysfunction or underfunction, and is roughly grouped under type 1 (insulin-dependent, some are slowly progressive diabetes starting from non-insulin-dependent) diabetes and type 2 (non-insulin-dependent) diabetes. Further, type 2 diabetes is categorized into those characterized by obesity with hyperinsulinism and insulin resistance (insulin functional disorder), characterized by non-obesity with insulin secretion disorder, and those with both disorders.

The treatment method for diabetes includes exercise-approach, diet-approach, and pharmaceutical approach, and in conducting any approach, keeping track of patient's blood-sugar level measurement values such as blood pressure and blood-sugar level; energy amount consumed in exercises; meal intake amount; and the like is necessary. Particularly, accuracy is required for conducting management of meal intake timing and intake amount, management of blood-sugar level measurement timing, management of changes in blood-sugar level, prediction of a future blood-sugar level, and a grasp on the energy amount by nutrient to be taken.

Thus, in exercise-approach, patients have been carrying out a work of consuming energy as prescribed by a medical institute by running, swimming, or an exercise using an exercise assisting device such as treadmills, and recording in handwriting or so the consumed energy amount. Also, in meal-approach, patients themselves have been carrying out a work of estimating the food material and its weight visually, calculating the energy amount by nutrient based on the Tables of Food Composition, and recording by handwriting or so. Additionally, in realities, the measurement-timing for the blood-sugar level has been checked by using such records and ordinary clocks.

For such problems, in patent publication document 1, for example, a mobile terminal for self-health-management and a support system for self-health-management have been proposed, for supporting the exercise-approach and the meal-approach effectively and for reducing a burden on patients. Also, a health management system is disclosed in patent publication document 2, in which data on blood-sugar level is sent to a computer of a hospital by a mobile phone, and a corresponding prescription is automatically sent to the patient's mobile phone based on the measured blood-sugar level data sent.

However, the self-management support system in patent publication document 1 is, considered as large-sized and unsuitable for carrying along, mainly aimed for energy amount management by nutrient contained in food material, and without particular functions for patients to recognize easily the results from measuring and managing the blood-sugar level.

The health management system of patent publication document 2 is as well considered large-sized and unsuitable for carrying along, and prescriptions are just sent from computers to mobile phones of patients: measurement and management of blood-sugar level, and its results are unavailable for patients to conveniently and easily use for the exercise-approach and the meal-approach.

Additionally, although as a conventional blood-sugar monitoring device, the one including a monitoring unit, a data-accumulation unit, a time-management unit, and a display unit for displaying necessary information (numeral value, time, and the like) is known, its functions are just enough for displaying the blood-sugar level after completing the measurement and the blood-sugar level measured in the past along with the date and time of the measurement, and the display contents and display method are not effectively applicable to the maximum for patient's meal-approach and exercise-approach.

Patent Publication Document 1:

Japanese Laid-Open Patent Publication No. 2002-222263

Patent Publication Document 2:

Japanese Laid-Open Patent Publication No. 2003-180637

### DISCLOSURE OF INVENTION

### The Problem to be Solved by the Invention

Thus, an object of the present invention is to provide a mobile blood-sugar level monitoring device, in which management of blood-sugar level measurement timing, management of changes in blood-sugar level, prediction of a future blood-sugar level, and further, management of meal intake timing and intake amount, can be carried out accurately and easily, for assisting the meal-approach and the exercise-approach to be effective for diabetic patients, regardless of whether the patient is type 1 or type 2.

### Means for Solving the Problem

To solve the problems as noted in the above, the present invention provides a blood-sugar level monitoring device with functions to measure a blood-sugar level of an examinee and to manage the obtained measurement data, the device comprising:

a monitoring unit for measuring the blood-sugar level of the examinee;

a measurement-data accumulation unit for accumulating the measured blood-sugar level as measurement data;

a data arithmetic-processing unit for arithmetic-processing the measurement data;

a display device for displaying a result of the arithmetic-processing;

an output-device control-unit for controlling the display device;

a time-management unit for managing a measurement-timing for the blood-sugar level;

a control switch for controlling the monitoring unit, the measurement-data accumulation unit, the data arithmetic-processing unit, the display device, the output-device control-unit, and the time-management unit; and

an external data importing unit for importing external data on the blood-sugar level.

In the blood-sugar level monitoring device in accordance with the present invention, the data arithmetic-processing unit is advantageously provided with a means or function for creating a graph in which the measured blood-sugar level is plotted against time based on the measurement data; and the display device is advantageously provided with a means or function for displaying the graph.

Also, the data arithmetic-processing unit is advantageously provided with a means or function for creating a graph in which the measured blood-sugar level is plotted against elapsed time from immediately after a start of a breakfast, lunch, or dinner intake based on the measurement data; and the display device is advantageously provided with a means or function for displaying the graph.

The data arithmetic-processing unit is preferably provided with a period-changing means or function for changing the period of the time by the control switch.

Also, the measurement-data accumulation unit is preferably provided with a means or function for changing the accumulation period of the measurement data.

The external data is preferably a target blood-sugar level set by a medical doctor.

Also, the display device is preferably provided with a means or function for plotting the target blood-sugar level on the graph in cooperation with the data arithmetic-processing unit.

Also, the data arithmetic-processing unit is advantageously provided with a means or function for creating a smoothed blood-sugar response curve showing a tendency of the measured blood-sugar level against time based on the measurement data by a smoothing process using the least squares method or the Gaussian Kernel method, or by a smoothing process using the running median; and the display device is advantageously provided with a means or function for displaying the smoothed blood-sugar response curve on the graph.

Further, the data arithmetic-processing unit is advantageously provided with a means or function for creating a target blood-sugar response curve showing the target blood-sugar level against time based on the measurement data or external data, by a linear interpolation method, the Spline interpolation method, or the Lagrange interpolation method; and the display device is advantageously provided with a means or function for displaying the target blood-sugar response curve.

The measurement data to be assigned advantageously comprises three measured blood-sugar levels including at least a blood-sugar level at a meal-start and a blood-sugar level at two hours after meal.

Also, the measurement data to be assigned advantageously comprises a blood-sugar level at a meal-start, a peak blood-sugar level, a time period from the meal-start to the point when the peak blood-sugar level is obtained, and a blood-sugar level at two hours after the meal-start.

Also, the measurement data to be assigned advantageously comprises a blood-sugar level at a meal-start, a blood-sugar level at one hour after the meal-start, and a blood-sugar level at two hours after the meal-start.

The data arithmetic-processing unit is advantageously provided with a means or function for creating a target blood-sugar response curve showing a target blood-sugar level against time based on the measurement data accumulated in a predetermined period in the past, by a linear interpolation method, the Spline interpolation method, or the Lagrange interpolation method; and the display device is advantageously provided with a means or function for displaying the target blood-sugar response curve.

Also, the data arithmetic-processing unit is advantageously provided with a means or function for creating a target blood-sugar response curve showing a target blood-sugar level against time based on the external data, by a linear interpolation method, the Spline interpolation method, or the Lagrange interpolation method; and the display device is advantageously provided with a means or function for displaying the target blood-sugar response curve.

Also, the data arithmetic-processing unit is preferably provided with a means or function for calculating a predicted blood-sugar level after an elapse of a predetermined time period based on the measurement data accumulated in a certain time period in the past or on the latest measurement data; and the display device is preferably provided with a means or function for displaying the predicted blood-sugar level.

Also, the control switch advantageously includes a timing-input switch for inputting the measurement timing.

Further, the blood-sugar level monitoring device in accordance with the present invention preferably comprises an output unit for generating at least one selected from the group consisting of sound, vibration, and light; and the output-device control-unit is preferably provided with a means or function for controlling the output unit.

Also, in cooperation with the time-management unit, the display device advantageously displays the measurement timing or the time till the measurement timing; and the output unit advantageously warns by generating at least one selected from the group consisting of sound, vibration, and light.

The display device is preferably provided with a means or function for displaying the latest of the measured blood-sugar level by blinking or as a predetermined mark.

Also, the display device is preferably provided with a means or function for scrolling the displayed contents in the horizontal direction or in the vertical direction.

Further, the display device is advantageously provided with a means for rotating the displayed contents upside-down.

Also, the external data is preferably enabled to be imported to the external data importing unit through wired or wireless communication; and the external data is advantageously enabled to be imported to the external data importing unit via at least one of a personal computer and a memory card.

### EFFECT OF THE INVENTION

According to a blood-sugar level monitoring device of the present invention, management of meal intake timing and intake amount, management of blood-sugar level measurement timing, management of changes in blood-sugar level, and further prediction of a future blood-sugar level can be carried out accurately and joyfully. Thus, the blood-sugar level monitoring device of the present invention is very effective in assisting diabetic patients' meal-approach and exercise-approach.

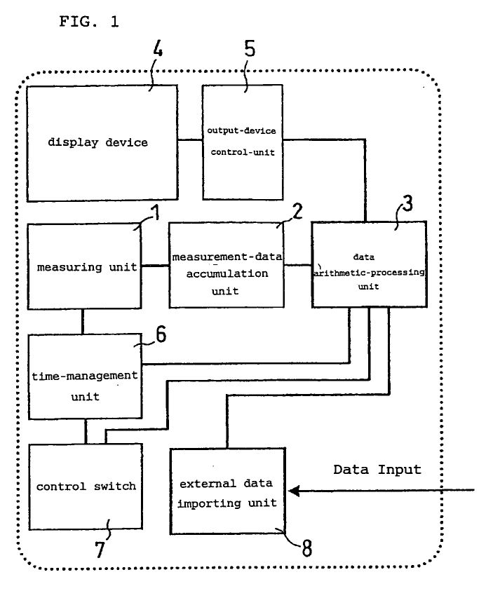

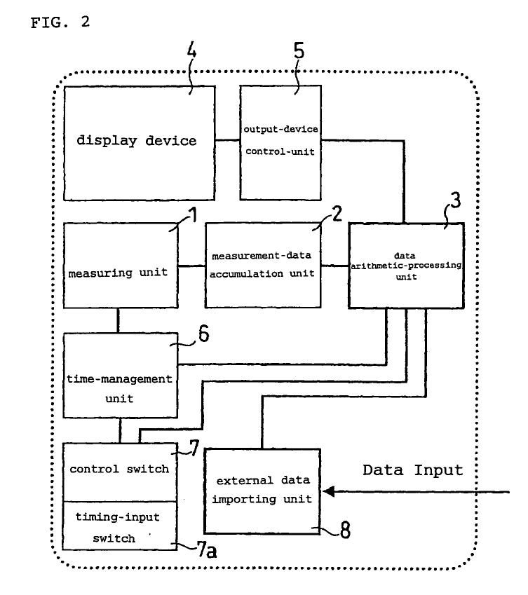

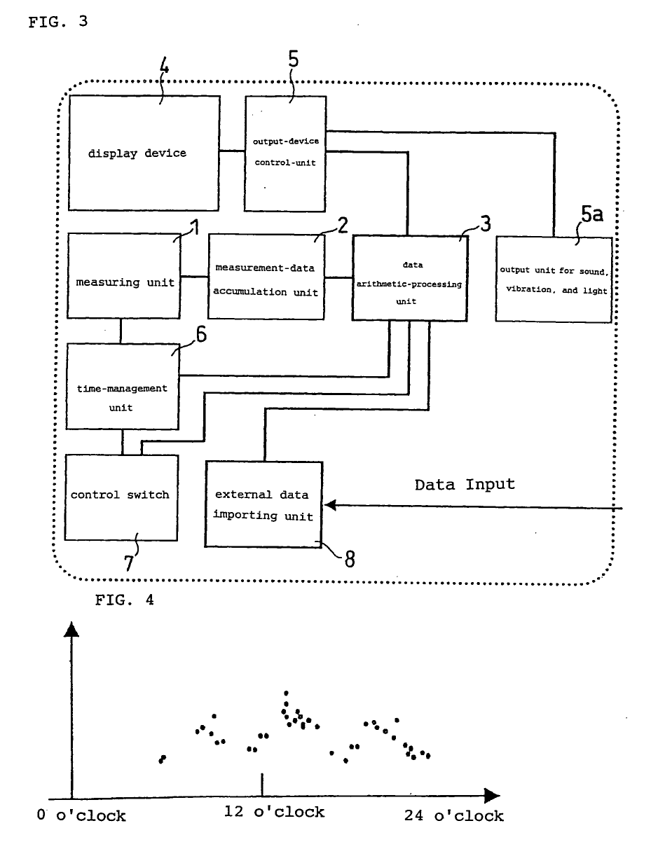

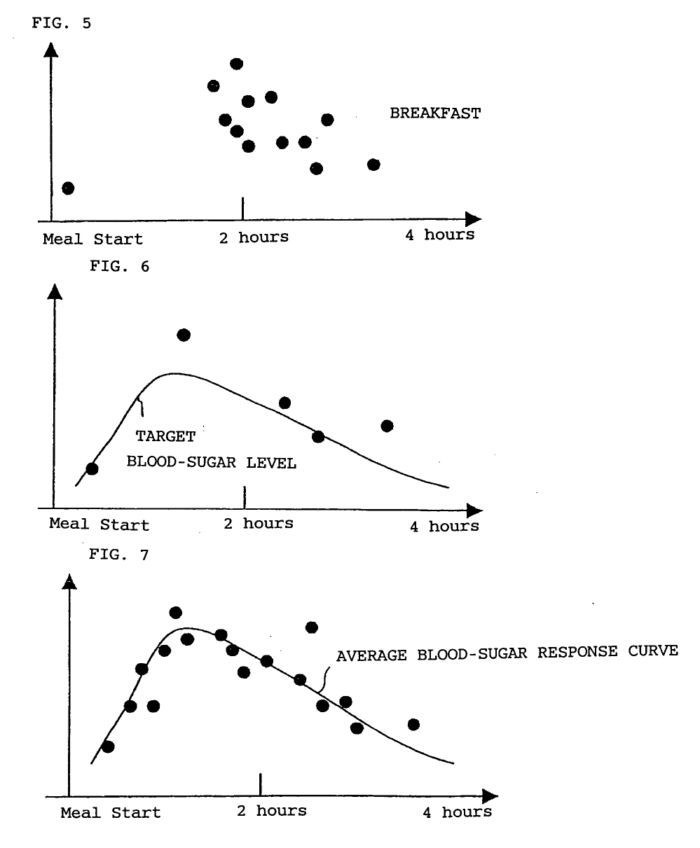

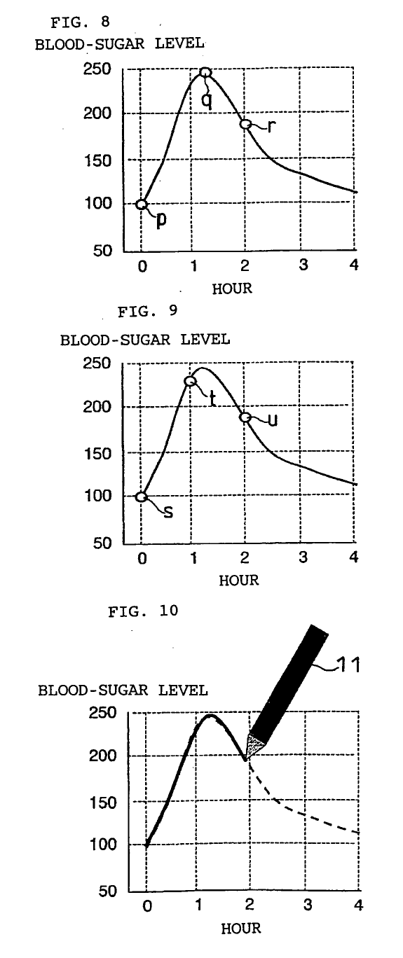

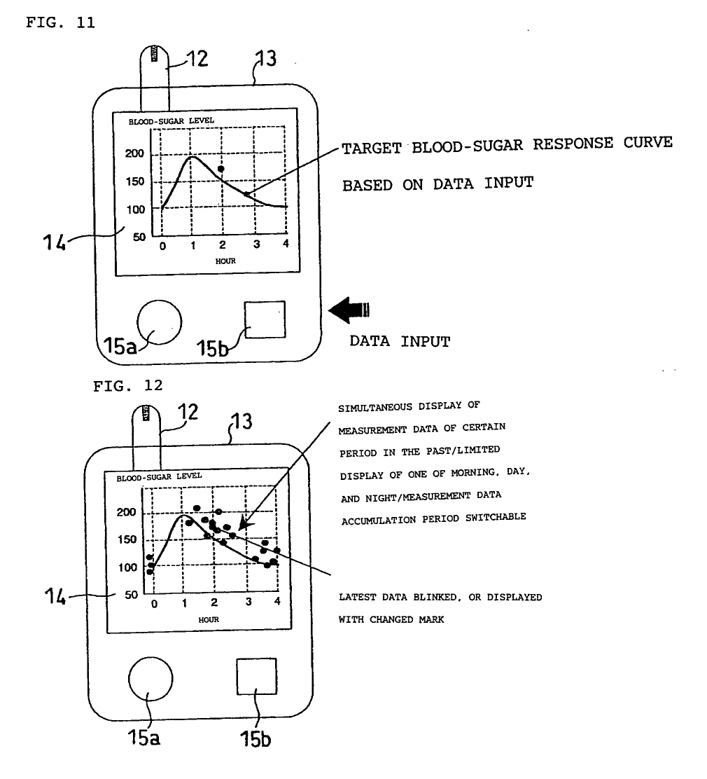

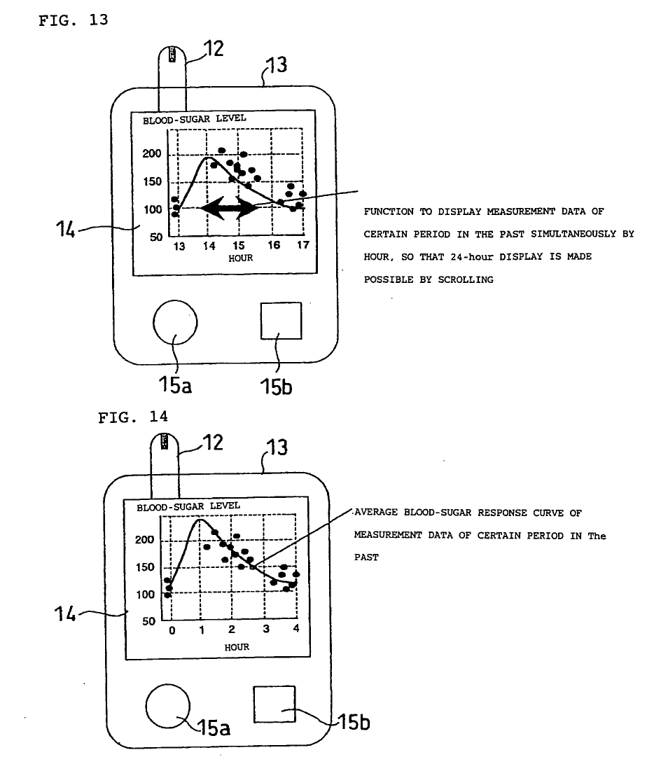

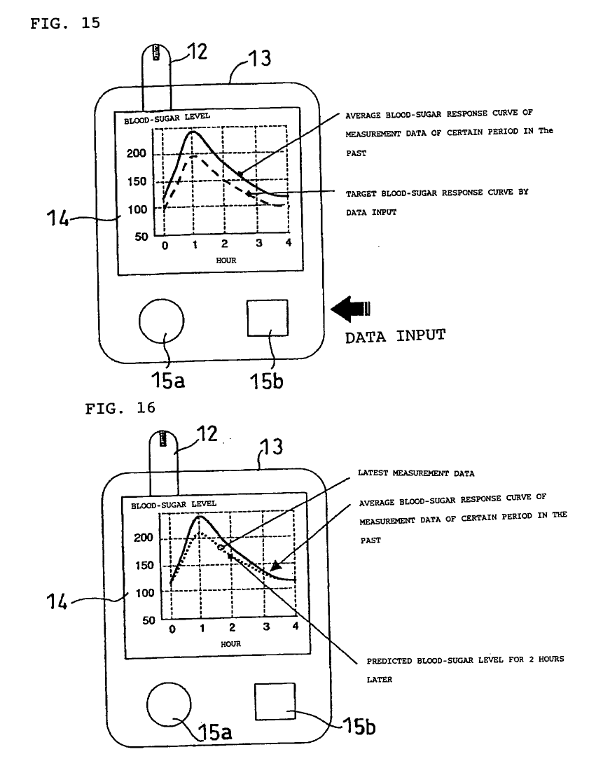

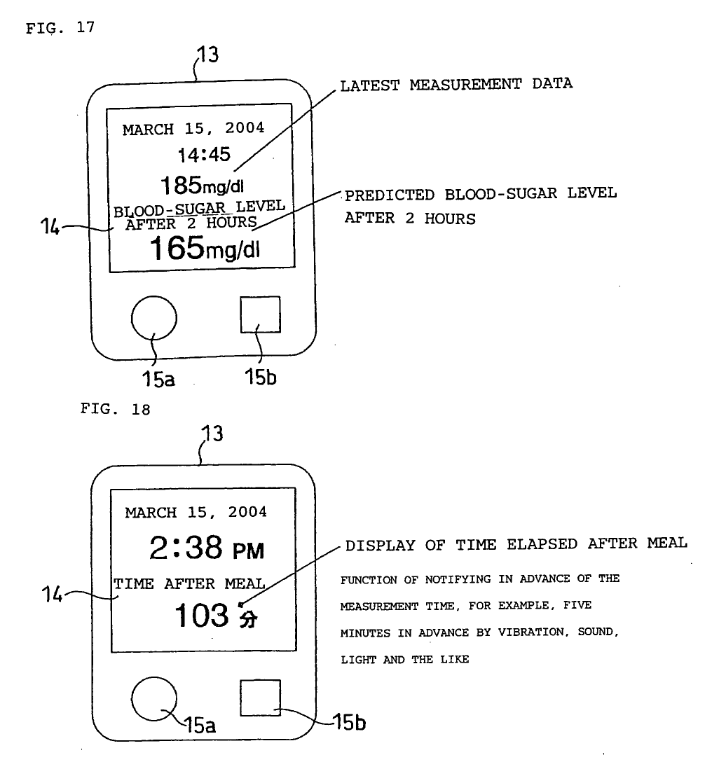

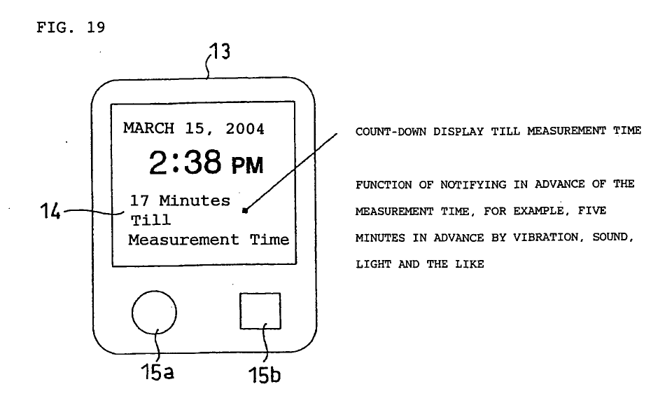

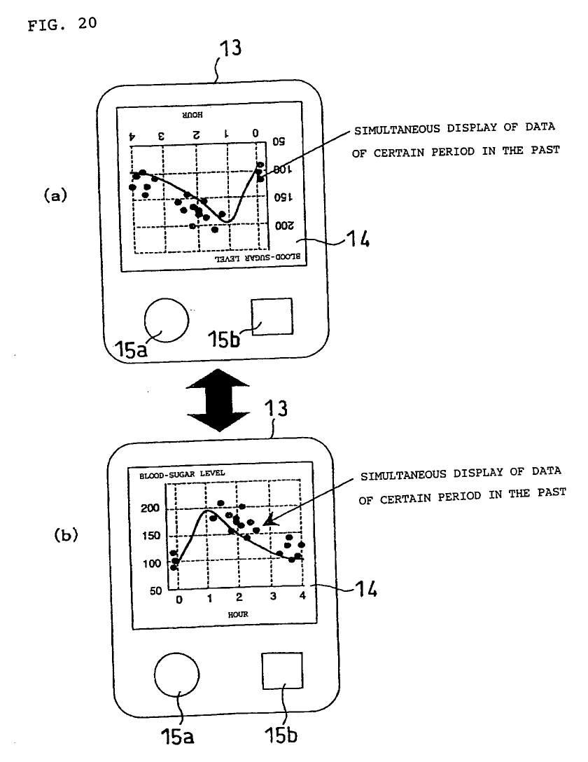

### BRIEF DESCRIPTION OF THE DRAWINGS

[FIG. 1] A block diagram illustrating a structure of a blood-sugar level monitoring device of the present invention.

[FIG. 2] A block diagram illustrating a structure of another blood-sugar level monitoring device of the present invention.

[FIG. 3] A block diagram illustrating a structure of still another blood-sugar level monitoring device of the present invention.

[FIG. 4] An example of a graph made by a data arithmetic-processing unit 3 based on measurement data.

[FIG. 5] Another example of a graph made by a data arithmetic-processing unit 3 based on measurement data.

[FIG. 6] Another example of a graph made by a data arithmetic-processing unit 3 based on measurement data.

[FIG. 7] Still another example of a graph made by a data arithmetic-processing unit 3 based on measurement data.

[FIG. 8] A graph including a target blood-sugar response curve obtained by a predetermined higher order approximation function, showing time and a blood-sugar level.

[FIG. 9] A graph including another target blood-sugar response curve obtained by a predetermined higher order approximation function, showing time and a blood-sugar level.

[FIG. 10] A diagram illustrating a manner in which a target blood-sugar response curve is inputted at a display device of an external data-input device with a pen 11.

[FIG. 11] A schematic diagram of a blood-sugar level monitoring device 13 in accordance with the present invention.

[FIG. 12] A schematic diagram of a blood-sugar level monitoring device 13 with another function in accordance with the present invention.

[FIG. 13] A schematic diagram of a blood-sugar level monitoring device 13 with still another function in accordance with the present invention.

[FIG. 14] A schematic diagram of a blood-sugar level monitoring device 13 with still another function in accordance with the present invention.

[FIG. 15] A schematic diagram of a blood-sugar level monitoring device 13 with still another function in accordance with the present invention.

[FIG. 16] A schematic diagram of a blood-sugar level monitoring device 13 with still another function in accordance with the present invention.

[FIG. 17] A schematic diagram of a blood-sugar level monitoring device 13 with still another function in accordance with the present invention.

[FIG. 18] A schematic diagram of a blood-sugar level monitoring device 13 with still another function in accordance with the present invention.

[FIG. 19] A schematic diagram of a blood-sugar level monitoring device 13 with still another function in accordance with the present invention.

[FIG. 20] A schematic diagram of a blood-sugar level monitoring device 13 with still another function in accordance with the present invention.

### BEST MODE FOR CARRYING OUT THE INVENTION

A structure of a blood-sugar level monitoring device in accordance with the present invention is explained with reference to the drawings. FIG. 1 is a block diagram illustrating a structure of a blood-sugar level monitoring device in accordance with the present invention.

As shown in FIG. 1, a blood-sugar level monitoring device in accordance with the present invention comprises a monitoring unit 1 for measuring a blood-sugar level, a measurement-data accumulation unit 2 for accumulating measurement data, a data arithmetic-processing unit 3 for arithmetically processing measurement data, a display device 4 for displaying a result of the arithmetic process, an output-device control-unit 5 for controlling the display device 4, and a time-management unit 6 for managing a measurement timing and the like. The blood-sugar level monitoring device further comprises: a control switch 7 for controlling the monitoring unit 1, the measurement-data accumulation unit 2, the data arithmetic-processing unit 3, the display device 4, the output-device control-unit 5, and the time-management unit 6; and an external data importing unit 8 capable of importing the external data on blood-sugar level.

The monitoring unit 1 is a part for measuring a blood-sugar level of an examinee (patient) by using a biosensor and the like. For the biosensor, the one conventionally used for measuring a blood-sugar level may be used. For example, a chip biosensor described in Japanese Laid-Open Patent Publication No. Hei 2-062952 is preferably used.

The biosensor described in the above Publication is made by forming an electrode system comprising a working electrode, a counter electrode, and a reference electrode on an insulating base plate by a method of screen printing or the like; and forming an enzyme reaction layer contacting the electrode system and including a hydrophilic polymer, an oxidoreductase (glucose oxidase), and an electron acceptor.

When a sample solution (blood) including a substrate (glucose) is dropped onto the enzyme reaction layer of the biosensor thus made, the enzyme reaction layer is dissolved and the glucose oxidase and substrate are reacted, thereby reducing the electron acceptor. After the completion of the enzyme reaction, the reduced electron acceptor is oxidized electrochemically, and the concentration of the substrate (blood-sugar level) in the sample solution can be determined from a value of oxidation current obtained at this time.

Then, the measurement-data accumulation unit 2 accumulates the measured blood-sugar level obtained by the monitoring unit 1 as measurement data. Thus, for the measurement-data accumulation unit 2, a storage medium capable of reading/writing such as a semiconductor memory and the like may be used.

The data arithmetic-processing unit 3 is the most characteristic part in a blood-sugar level monitoring device in accordance with the present invention, and is particularly characterized by its arithmetic processing method.

The data arithmetic-processing unit 3 is firstly capable of arithmetically processing the measurement data and/or the external data, and creating (mapping) a graph from the result of the arithmetic process and the like. Secondly, the data arithmetic-processing unit 3 calculates a future target blood-sugar level, a predicted blood-sugar level, a measurement timing, and the like from the measurement data and external data.

Functions of the data arithmetic-processing unit 3 are described in detail in the following. For the data arithmetic-processing unit 3, may be used is a microprocessor or the like including a CPU and a memory, for memorizing and executing programs and applications realizing various functions described in the specification of the present invention.

Then, the display device 4 displays the result of the arithmetic process by the data arithmetic-processing unit 3, the graph showing the results, the mean values and interpolation data of the measurement data, a predicted blood-sugar level, measurement timing, and the like. Patients and further medical doctors thus can compare the data with external data and the like, grasp the measurement data of blood-sugar levels from various viewpoints, and make use of the measurement data of blood-sugar levels for meal-approach and exercise-approach for diabetes. An ordinary liquid crystal display device may be used for the display device 4.

The output-device control-unit 5 is for controlling the display device 4 and an output unit mentioned later. For the output-device control-unit 5, for example, an image signal processing IC and the like may be used.

The time-management unit 6 for managing the timing of a blood-sugar level measurement by the monitoring unit 1 plays a role to calculate and manage the measurement timing in cooperation with the data arithmetic-processing unit 3, and to notify the patient the measurement timing by alarming in cooperation with the display device 4 and the output unit to be mentioned later. For the time-management unit 6, for example, a clock including a quarts oscillator may be used.

The control switch 7 is for controlling the monitoring unit 1, the measurement-data accumulation unit 2, the data arithmetic-processing unit 3, the display device 4, the output-device control-unit 5, and the time-management unit 6.

For example, via the control switch, patients and medical doctors measure a blood-sugar level with the monitoring unit 1, select the measurement data (for example, the measurement data of a certain period in the past) to be accumulated in the measurement-data accumulation unit 2, and let the data arithmetic-processing unit 3 arithmetically process the measurement data and external data.

Additionally, controlling the display device 4 can be effective for exhibiting functions of selecting a particular portion such as the latest measured blood-sugar level to be blinked or displayed with a predetermined mark, scrolling the displayed content such as a graph displayed in the display device 4 in the horizontal direction (or the vertical direction), and rotating (inversing) 180 degrees.

The external data importing unit 8 is for importing external data on blood-sugar level.

The external data include, blood-sugar measurement data (calorie vs. blood-sugar level) by regular meal, blood-sugar measurement data (calorie vs. blood-sugar level) by commercially available diabetic meal, a target blood-sugar level set by a medical doctor for a patient, accumulation of the past measurement data of the patient, consumed calories, meal calories, the PFC ratio (ratio between protein, fat, and carbohydrate), meal menu, the heart rate appropriate for the exercise-approach, an exercise amount (number of steps), and other various data needed by diabetic patients.

As shown in FIG. 2, the control switch 7 is preferably provided with a timing-input switch for entering a measurement timing. This is used for diabetic patients to enter the time of the meal-start in the blood-sugar level monitoring device.

The blood-sugar level can be measured immediately before meal, and the time can be entered as a meal-start time.

Further, as shown in FIG. 3, an output unit 5a for generating at least one selected from the group consisting of sound, vibration, and light is preferably connected to the output-device control-unit 5, and the output-device control-unit 5 preferably has a means for controlling the output unit 5a. According to this, a measurement-timing for a blood-sugar level after meal can be notified to the patient. Such measurement timing is not only limited to the time for the blood-sugar level measurement after meal, for example, and can be set to 5 minutes before the time of the measurement.

Also, at least one of sound, vibration, and light can be generated for every determined time period for example every 2 hours, by the management of the time-management unit 6.

Basic functions of the data arithmetic-processing unit 3 are briefly explained.

First, the data arithmetic-processing unit 3 preferably has a means or function for creating a graph plotting the measured blood-sugar level against time based on the measurement data, and is able to display the graph on the display device 4.

FIG. 4 shows an example of the graph thus created. FIG. 4 is a graph obtained by plotting time on the horizontal axis, and plotting the measured blood-sugar level on the vertical axis based on the measurement data.

Also, the data arithmetic-processing unit 3 preferably has a means or function for creating a graph plotting the measured blood-sugar level against time period from immediately after a start of a breakfast, lunch, or dinner intake (meal-start time) based on the measurement data, and is able to display the graph on the display device 4.

FIG. 5 shows an example of the graph thus created. FIG. 5 is a graph obtained by plotting the measured blood-sugar level on the vertical axis and time elapsed from immediately after the breakfast intake (meal-start time) on the horizontal axis based on the measurement data.

Also, the data arithmetic-processing unit 3 preferably has a means or function for plotting a target blood-sugar response curve on the graph, the curve illustrating a target blood-sugar level obtained by for example the Spline interpolation method based on the external data imported from the external data importing unit 8, such as for example a target blood-sugar level set by a medical doctor for the patient. Based on such graph, the patient can check if his own current blood-sugar level is good or not immediately.

FIG. 6 shows an example of the graph thus created. In FIG. 6, time elapsed from immediately after the breakfast intake (meal-start time) is plotted on the horizontal axis, and the measured blood-sugar level is plotted on the vertical axis based on the measurement data, and further, a target blood-sugar response curve showing a target blood-sugar level set by for example medical doctors or the like is shown. Based on such graph, the patient can check if his/her blood-sugar level is good or not against the elapsed time after the meal-start.

Further, the data arithmetic-processing unit 3 preferably has a means or function for creating a smoothed blood-sugar response curve showing tendencies for the measured blood-sugar level against time by obtaining a higher order approximation function based on the measurement data with a smoothing process using the least squares method or the Gaussian Kernel method, or a smoothing process using running median, and as shown in FIG. 7, the display device 4 preferably shows the curve on the graph.

For the higher order approximation function, for example, aX4+bX3+cX2+dX+e may be mentioned. The period and number of the measurement data for the smoothing and interpolation processes can be set arbitrary.

A creation of a target blood-sugar response curve, an important new function of the data arithmetic-processing unit 3, is explained next.

The data arithmetic-processing unit 3 preferably has a means or function for creating a target blood-sugar response curve made up by a target blood-sugar level against time, based on the measurement data accumulated in the measurement-data accumulation unit 2 and the measurement data imported as external data, by for example a linear interpolation method, the Spline interpolation method, or the Lagrange interpolation method, and the display device 4 preferably displays the target blood-sugar response curve.

For the higher order approximation function, for example, aX4+bX3+cX2+dX+e may be mentioned.

The measurement data used for creating a target blood-sugar response curve is explained here. The curves shown in FIGS. 9 and 8 are a target blood-sugar response curve obtained by the Spline interpolation method, showing time and blood-sugar level.

First, as shown in FIG. 8, the measurement data to be assigned preferably include three measured blood-sugar levels including at least a blood-sugar level p at a meal-start and a blood-sugar level r at 2 hours after the meal-start. More particularly, in addition to the blood-sugar levels p and r, a peak blood-sugar level q, and time taken for obtaining the peak blood-sugar level q are preferably included.

This is because the peak blood-sugar level is important for diagnosis of diabetes, not only to patients but also from the viewpoint of medical doctors.

Also, as shown in FIG. 9, the measurement data to be used may include a blood-sugar level s at a meal-start, a blood-sugar level t at 1 hour after the meal-start, and a blood-sugar level u at 2 hours after the meal-start.

In this case, the peak blood-sugar level is not necessarily measured, and it is preferable in the sense that a burden on patients with regard to blood-sugar level measurement can be lessened.

Although it is assumed in the above that the processing using a linear interpolation method, the Spline interpolation method, or the least squares method is carried out by the data arithmetic-processing unit 3 of the blood-sugar level monitoring device in accordance with the present invention, the target blood-sugar response curve by a higher order approximation function may be displayed based on coefficient data, by calculating the coefficient from the measurement data using the Spline interpolation method and the least squares method at an external data-input device (PC, PDA, or specialized device), and sending the obtained coefficient data to the blood-sugar level monitoring device again.

Also, as shown in FIG. 10, the target blood-sugar response curve may be entered at a display device such as a touch panel of the external data-input device (PC, PDA, or specialized device), with a pen 11 (or a mouse) arbitrary, and the data can be sent to the blood-sugar level monitoring device and displayed. In such case, for example, a target blood-sugar response curve can be created on the spot while a medical doctor is giving an explanation to the patient face to face upon patient's visit to a medical institute.

In the following, by referring to the drawings, functions of the data arithmetic-processing unit 3 of the blood-sugar level monitoring device in accordance with the present invention, and the contents to be displayed in the display device 4 based on the functions are described as embodiments.

### EMBODIMENT 1

FIG. 11 is a schematic diagram of a blood-sugar level monitoring device 13 in accordance with the present invention. A biosensor 12 is inserted into the blood-sugar level monitoring device 13: a display device 14 shows a point indicating the measured blood-sugar level and a target blood-sugar response curve obtained by the Spline interpolation method with a data arithmetic-processing unit 3, based on data from an external data importing unit 8.

Also, with control switches 15a and 15b, a monitoring unit 1 can be controlled, a kind and a period of the measurement data to be used for the arithmetic process can be selected, and the measurement data can be entered manually.

The target blood-sugar response curve can be created by various methods as described above. By showing both the target blood-sugar response curve and the latest measurement data, data can be provided for use by patients and medical doctors.

In FIG. 11, the horizontal axis indicates time, setting a meal-start as the starting point. Such display can be shown by breakfast, lunch, and dinner.

### EMBODIMENT 2

FIG. 12 is a schematic diagram of another blood-sugar level monitoring device 13 in accordance with the present invention. A biosensor 12 is inserted into the blood-sugar level monitoring device 13: a display device 14 shows points indicating blood-sugar levels of the past measurement data accumulated in a measurement-data accumulation unit 2, and a target blood-sugar response curve obtained by the Spline interpolation method with a data arithmetic-processing unit 3, based on data from an external data importing unit 8.

Also, with control switches 15a and 15b, a monitoring unit 1 can be controlled, a kind and a period of the measurement data to be used for the arithmetic process can be selected, and the measurement data can be entered manually.

The target blood-sugar response curve can be created by the various methods described in the above. Also, for the latest measurement data, blinking, a changed mark, and the like are used for easier recognition.

In FIG. 12, the horizontal axis indicates time, setting a meal-start as the starting point. Such display can be shown by breakfast, lunch, and dinner.

### EMBODIMENT 3

FIG. 13 is a schematic diagram of another blood-sugar level monitoring device 13 in accordance with the present invention. A biosensor 12 is inserted into the blood-sugar level monitoring device 13: a display device 14 can show points indicating blood-sugar levels of the past measurement data accumulated in a measurement-data accumulation unit 2 by a period of 24 hours. Also shown is a target blood-sugar response curve obtained by the Spline interpolation method with a data arithmetic-processing unit 3 based on data from an external data importing unit 8 (This can be omitted.). However, since displaying the measurement data by the period of 24 hours reduces viewability, it is effective to provide the display device 4 with a function of scrolling in the horizontal axis (arrow) direction so that the measurement data of specific 2 to 6 hours can be displayed.

As in the above case, with control switches 15a and 15b, a monitoring unit 1 can be controlled, a kind and a period of the measurement data to be used for the arithmetic process can be selected, and the measurement data can be entered manually. The target blood-sugar response curve can be created by the various methods described in the above. For the latest measurement data, blinking and a changed mark may be used for easier recognition.

### EMBODIMENT 4

FIG. 14 is a schematic diagram of another blood-sugar level monitoring device 13 in accordance with the present invention. A biosensor 12 is inserted into the blood-sugar level monitoring device 13: the display device 14 shows points indicating blood-sugar levels of the past measurement data accumulated in a measurement-data accumulation unit 2, and a smoothed blood-sugar response curve obtained by the least squares method with a data arithmetic-processing unit 3 based on for example measurement data accumulated in a predetermined period in the past.

In this case as well, since displaying the measurement data of 24 hours reduces viewability, a function of scrolling in the horizontal axis (arrow) direction can be provided so that the display device 4 can display the measurement data of specific 2 to 6 hours. Also, with control switches 15a and 15b, a monitoring unit 1 can be controlled, a kind and a period of the measurement data to be used for the arithmetic process can be selected, and the measurement data can be entered manually. The smoothed blood-sugar response curve may be created by various methods as described above, and for the latest measurement data, blinking, a changed mark, and the like can be used as well for easier recognition.

### EMBODIMENT 5

FIG. 15 is a schematic diagram of another blood-sugar level monitoring device 13 in accordance with the present invention. A biosensor 12 is inserted into the blood-sugar level monitoring device 13: a display device 14 shows a smoothed blood-sugar response curve and a target blood-sugar response curve obtained by the least squares method with the data arithmetic-processing unit 3 based on for example measurement data accumulated in a predetermined period in the past. This embodiment has merits, in view of assisting the target-management by patient himself in the treatment.

In this case as well, since displaying the measurement data of 24 hours reduces viewability, a function of scrolling in the horizontal axis (arrow) direction can be provided so that the display device 4 can display the measurement data of specific 2 to 6 hours. Also, with control switches 15a and 15b, a monitoring unit 1 can be controlled, a kind and a period of the measurement data to be used for the arithmetic process can be selected, and the measurement data can be entered manually. The Spline interpolation curve can be created by various methods as mentioned above. For the latest measurement data, blinking, a changed mark, and the like can be used for easier recognition.

### EMBODIMENT 6

FIG. 16 is a schematic diagram of another blood-sugar level monitoring device 13 in accordance with the present invention. A biosensor 12 is inserted into the blood-sugar level monitoring device 13: the display device 14 shows a point indicating a latest measured blood-sugar level, and a smoothed blood-sugar response curve obtained by carrying out the least squares method with the data arithmetic-processing unit 3, based on the measurement data accumulated in a predetermined period in the past. Further, a predicted blood-sugar level for after an elapse of 2 hours is calculated from the smoothed blood-sugar response curve and the measured blood-sugar level, and the predicted blood-sugar level is displayed. This embodiment has merits in that the patient achieves visual comprehension in his management for the target.

In this case as well, since displaying the measurement data of 24 hours reduces viewability, a function of scrolling in the horizontal axis (arrow) direction can be provided so that the display device 4 can display the measurement data of specific 2 to 6 hours. Also, with control switches 15a and 15b, a blood-sugar level monitoring device 1 can be controlled, a kind and a period of the measurement data used for the arithmetic process can be selected, and the measurement data can be entered manually. The smoothed blood-sugar response curve may be created by various methods as described above. For the latest measurement data and the predicted blood-sugar level, blinking and a changed mark can be used as well for easier recognition.

### EMBODIMENT 7

FIG. 17 is a schematic diagram of another blood-sugar level monitoring device 13 in accordance with the present invention. The biosensor is omitted. A display device 14 shows, as textual information, the latest measured blood-sugar level and a predicted blood-sugar level for after an elapse of 2 hours calculated from the smoothed blood-sugar response curve and the measured blood-sugar level as mentioned in the above Embodiment 7. This embodiment has merits in that it is useful in giving medical doctor's guidance for daily life for a treatment, with regard to giving instruction on a blood-sugar level after an elapse of specific time such as after 2 hours. Additionally, time and date can be indicated by a time-management unit 6 as well.

In this case as well, with control switches 15a and 15b, a blood-sugar level monitoring device 1 can be controlled, a kind and a period of the measurement data to be used for the arithmetic process can be selected, and the measurement data can be entered manually. The smoothed blood-sugar response curve may be created by various methods as described above. The texts of the latest measurement data and the predicted blood-sugar level can be allowed to blink for easier recognition.

### EMBODIMENT 8

FIG. 18 is a schematic diagram of another blood-sugar level monitoring device 13 in accordance with the present invention. The biosensor is omitted. A display device 14 shows, for example when a blood-sugar level is to be measured every 2 hours, time elapsed after a meal by a management of a time-management unit 6.

Also, as shown in FIG. 19, when a measurement time approaches, an alarm before 5 minutes is given by vibration, sound, or light by for example providing an output unit at an output information controlling unit 5 (not shown).

Such embodiments have merits in that a blood-sugar level can be measured at a specified time appropriately. Additionally, date and time can be indicated by a time-management unit 6 as well.

In this case as well, with control switches 15a and 15b, a monitoring unit 1 can be controlled, a kind and a period of the measurement data to be used for the arithmetic process can be selected, and the measurement data can be entered manually. Also, texts for the elapsed time after a meal can be blinked for easier recognition.

### EMBODIMENT 9

FIG. 20 is a schematic diagram of another blood-sugar level monitoring device 13 in accordance with the present invention. The biosensor is omitted. A display device 14 shows measurement data of a certain period of time in the past by points and a line, and has a function to rotate the display 180 degrees, upside-down, from (b) to (a) as shown.

With such function, when a patient is to receive a counseling from a medical doctor at a medical institute, a preferred display contents can be seen while they are in face-to-face.

External data can be imported to the external data importing unit 8, from a device such as external personal computers, by any of wired communication such as memory card or cable, and IR and RF wireless communication. There is no particular limitation on these.

Also, a blood-sugar level monitoring device in accordance with the present invention can be provided, in addition to the functions for managing the blood-sugar level as mentioned in the above, functions of providing the following to the patient from external data: the blood-sugar measurement data on regular meal (calorie vs. blood-sugar level), the blood-sugar measurement data on commercially available diabetes meal (calorie vs. blood-sugar level), the target blood-sugar level set by a medical doctor for a patient, accumulation of the patient's past measurement data, consumed calorie, meal calorie, PFC ratio, meal menu, the heart rate appropriate for exercise-approach, and other various data necessary for diabetic patients with relative to the measurement data. Further, functions can be provided for notifying meal timing and exercise timing, and for notifying necessity or not of meal and exercise.

### INDUSTRIAL APPLICABILITY

A blood-sugar level monitoring device in accordance with the present invention can be carried along, and enables management of meal intake timing and intake amount, management of blood-sugar level measurement timing, management of changes in blood-sugar level, and further a prediction of a future blood-sugar level, precisely and easily for diabetic patients who are conducting exercise-approach or meal-approach in their daily lives away from medical institutes. Therefore, with an increase in diabetic patients, the device can be used suitably in diabetes treatment by medical doctors and medical institutes.

1. A blood-sugar level monitoring device with functions to measure a blood-sugar level of an examinee and to manage obtained measurement data, the device comprising:
a monitoring unit for measuring the blood-sugar level of said examinee;
a measurement-data accumulation unit for accumulating the measured blood-sugar level as measurement data;
a data arithmetic-processing unit for arithmetically processing said measurement data;
a display device for displaying a result of said arithmetic-processing;
an output-device control-unit for controlling said display device;
a time-management unit for managing a measurement-timing for said blood-sugar level;
a control switch for controlling said monitoring unit, said measurement-data accumulation unit, said data arithmetic-processing unit, said display device, said output-device control-unit, and said time-management unit; and
an external data importing unit for importing external data on the blood-sugar level.

2. The blood-sugar level monitoring device in accordance with claim 1,
wherein said data arithmetic-processing unit is provided with a means for creating a graph in which said measured blood-sugar level is plotted against time based on said measurement data; and
said display device is provided with a means for displaying said graph.

3. The blood-sugar level monitoring device in accordance with claim 1,
wherein said data arithmetic-processing unit is provided with a means for creating a graph in which said measured blood-sugar level is plotted against elapsed time from immediately after a start of a breakfast, lunch, or dinner intake based on said measurement data; and
said display device is provided with a means for displaying said graph.

4. The blood-sugar level monitoring device in accordance with claim 2, wherein said data arithmetic-processing unit is provided with a period-changing means for changing a period of said time by said control switch.

5. The blood-sugar level monitoring device in accordance with claim 1, wherein said measurement-data accumulation unit is provided with a means for changing an accumulation period of said measurement data.

6. The blood-sugar level monitoring device in accordance with claim 1, wherein said external data is a target blood-sugar level set by a medical doctor.

7. The blood-sugar level monitoring device in accordance with claim 6, wherein said display device is provided with a means for plotting said target blood-sugar level on said graph in cooperation with said data arithmetic-processing unit.

8. The blood-sugar level monitoring device in accordance with claim 1,
wherein said data arithmetic-processing unit is provided with a means for creating a smoothed blood-sugar response curve showing a tendency of said measured blood-sugar level against time based on said measurement data; and
said display device is provided with a means for displaying the smoothed blood-sugar response curve on said graph.

9. The blood-sugar level monitoring device in accordance with claim 1,
wherein said data arithmetic-processing unit is provided with a means for creating a target blood-sugar response curve showing a target blood-sugar level against time based on said measurement data or external data; and
said display device is provided with a means for displaying said target blood-sugar response curve.

10. The blood-sugar level monitoring device in accordance with claim 9, wherein said measurement data to be assigned comprises three measured blood-sugar levels including at least a blood-sugar level at a meal-start and a blood-sugar level at two hours after the meal-start.

11. The blood-sugar level monitoring device in accordance with claim 9, wherein said measurement data to be assigned comprises a blood-sugar level at a meal-start, a peak blood-sugar level, a time period from the meal-start to the point when the peak blood-sugar level is obtained, and a blood-sugar level at two hours after the meal-start.

12. The blood-sugar level monitoring device in accordance with claim 9, wherein said measurement data to be assigned comprises a blood-sugar level at a meal-start, a blood-sugar level at one hour after the meal-start, and a blood-sugar level at two hours after the meal-start.

13. The blood-sugar level monitoring device in accordance with claim 1,
wherein said data arithmetic-processing unit is provided with a means for creating a target blood-sugar response curve showing a target blood-sugar level against time based on the measurement data accumulated in a predetermined period in the past; and
said display device is provided with a means for displaying said target blood-sugar response curve.

14. The blood-sugar level monitoring device in accordance with claim 1,
wherein said data arithmetic-processing unit is provided with a means for creating a target blood-sugar response curve showing a target blood-sugar level against time based on said external data; and
said display device is provided with a means for displaying said target blood-sugar response curve.

15. The blood-sugar level monitoring device in accordance with claim 1,
wherein said data arithmetic-processing unit is provided with a means for calculating a predicted blood-sugar level after an elapse of a predetermined time period based on the measurement data accumulated in a certain time period in the past or on latest measurement data; and
said display device is provided with a means for displaying said predicted blood-sugar level.

16. The blood-sugar level monitoring device in accordance with claim 1, wherein said control switch includes a timing-input switch for inputting said measurement timing.

17. The blood-sugar level monitoring device in accordance with claim 1, further comprising an output unit for generating at least one selected from the group consisting of sound, vibration, and light, wherein said output-device control-unit is provided with a means for controlling said output unit.

18. The blood-sugar level monitoring device in accordance with claim 17,
wherein said display device is provided with a means for displaying said measurement timing or a time till said measurement timing in cooperation with said time-management unit; and
said output unit is provided with a means for warning by generating at least one selected from the group consisting of sound, vibration, and light.

19. The blood-sugar level monitoring device in accordance with claim 1, wherein said display device is provided with a means for displaying a latest of said measured blood-sugar levels by blinking or as a predetermined mark.

20. The blood-sugar level monitoring device in accordance with claim 1, wherein said display device is provided with a means for scrolling displayed contents in a horizontal direction or a vertical direction.

21. The blood-sugar level monitoring device in accordance with claim 1, wherein said display device is provided with a means for rotating the displayed contents upside-down.

22. The blood-sugar level monitoring device in accordance with claim 1, wherein said external data can be imported to said external data importing unit through wired or wireless communication.

23. The blood-sugar level monitoring device in accordance with claim 1, wherein said external data can be imported to said external data importing unit via at least one of a personal computer and a memory card.
